(19) United States
(12) Patent Application Publication
(10) Pub. No.: US 2022/0022795 A1
(43) Pub. Date: Jan. 27, 2022

(54) PHYSIOLOGICAL SIGNAL SENSING SYSTEM AND METHOD

(71) Applicant: Industrial Technology Research Institute, Hsinchu (TW)

(72) Inventors: Kuang-Ching Fan, Hsinchu County (TW); Heng-Yin Chen, Hsinchu County (TW); Yun-Yi Huang, Pingtung County (TW); Yi-Cheng Lu, Hsinchu City (TW); Tzu-Hao Yu, Yilan County (TW)

(73) Assignee: Industrial Technology Research Institute, Hsinchu (TW)

(21) Appl. No.: 17/219,900

(22) Filed: Apr. 1, 2021

Related U.S. Application Data
(60) Provisional application No. 63/055,848, filed on Jul. 23, 2020.

(51) Int. Cl. A61B 5/277; A61B 5/313; A61B 5/308; A61B 5/31; A61B 5/00; A61B 5/27

(52) U.S. Cl. CPC A61B 5/277; A61B 5/313; A61B 5/308; A61B 2560/04; A61B 5/7225; A61B 5/27; A61B 5/31

(57) ABSTRACT

Provided are a physiological signal sensing system and a physiological signal sensing method. The physiological signal sensing system includes a physiological signal sensing apparatus, a variation sensing apparatus, and a signal processing apparatus. The physiological signal sensing apparatus is disposed on a fabric to sense and provide physiological signals of an organism. The physiological signal sensing apparatus includes capacitive coupling devices. The variation sensing apparatus is disposed on the fabric and includes a distance sensing device to sense a distance between the physiological signal sensing apparatus and the organism, and provide a first capacitance variation signal according to the distance. The signal processing apparatus is coupled to the physiological signal sensing apparatus and the variation sensing apparatus to receive the physiological signals and the first capacitance variation signal and correct the physiological signals according to the first capacitance variation signal to obtain corrected physiological signals.

### CROSS-REFERENCE TO RELATED APPLICATION

This application claims the priority benefits of U.S. provisional application Ser. No. 63/055,848, filed on Jul. 23, 2020 and Taiwan application serial no. 109146833, filed on Dec. 30, 2020. The entirety of each of the above-mentioned patent applications is hereby incorporated by reference herein.

### TECHNICAL FIELD

This application relates to a sensing system and method, and also relates to a physiological signal sensing system and method.

### BACKGROUND

With the advent of an aging society and the earlier onset of diseases of affluence, the number of elderly or patients who need to be cared for is gradually increasing. Therefore, various physiological signal sensing systems are developed to maintain the personal safety of the elderly or patients. Currently, for convenience and timeliness, a physiological signal sensing apparatus may be provided on clothing to sense the physiological signals of the human body.

However, the wearable physiological signal sensing apparatus tends to slide or shift when the user is moving, thus affecting sensing accuracy.

### SUMMARY

A physiological signal sensing system of the present application includes a physiological signal sensing apparatus, a variation sensing apparatus, and a signal processing apparatus. The physiological signal sensing apparatus is disposed on a fabric to sense and provide physiological signals of an organism. The physiological signal sensing apparatus includes capacitive coupling devices. The variation sensing apparatus is disposed on the fabric and includes a distance sensing device to sense a distance between the physiological signal sensing apparatus and the organism, and provide a first capacitance variation signal according to the distance. The signal processing apparatus is coupled to the physiological signal sensing apparatus and the variation sensing apparatus to receive the physiological signals and the first capacitance variation signal and perform a correction on the physiological signals according to the first capacitance variation signal to obtain corrected physiological signals.

A physiological signal sensing method of the present application includes the following steps. Physiological signals of an organism are sensed and provided via a physiological signal sensing apparatus disposed on a fabric. The physiological signal sensing apparatus includes capacitive coupling devices. A distance between the physiological signal sensing apparatus and the organism is sensed via a variation sensing apparatus including a distance sensing device disposed on the fabric, and a first capacitance variation signal is provided according to the distance. The physiological signals and the first capacitance variation signal are received via a signal processing apparatus coupled to the physiological signal sensing apparatus and the variation sensing apparatus, and a correction is performed on the physiological signals according to the first capacitance variation signal to obtain corrected physiological signals.

Several exemplary embodiments accompanied with figures are described in detail below to further describe the disclosure in details.

### BRIEF DESCRIPTION OF THE DRAWINGS

The accompanying drawings are included to provide further understanding, and are incorporated in and constitute a part of this specification. The drawings illustrate exemplary embodiments and, together with the description, serve to explain the principles of the disclosure.

FIG. 1A is a block diagram of the physiological signal sensing system of the first embodiment of the present application.

FIG. 1B is a flowchart of the physiological signal sensing method of the first embodiment of the present application.

FIG. 2A is a block diagram of the physiological signal sensing system of the second embodiment of the present application.

FIG. 2B is a flowchart of the physiological signal sensing method of the second embodiment of the present application.

FIG. 3A is a block diagram of the physiological signal sensing system of the third embodiment of the present application.

FIG. 3B is a flowchart of the physiological signal sensing method of the third embodiment of the present application.

FIG. 4A is a block diagram of the physiological signal sensing system of the fourth embodiment of the present application.

FIG. 4B is a flowchart of the physiological signal sensing method of the fourth embodiment of the present application.

FIG. 5A is a block diagram of the physiological signal sensing system of the fifth embodiment of the present application.

FIG. 5B is a flowchart of the physiological signal sensing method of the fifth embodiment of the present application.

FIG. 6 is a schematic diagram of the device configuration of the physiological signal sensing system of an embodiment of the present application.

FIG. 7A and FIG. 7B are respectively schematic diagrams of capacitive coupling devices of different embodiments of the present application.

FIG. 8A is a schematic diagram of the positional relationship between the physiological signal sensing apparatus and the fabric of an embodiment of the present application.

FIG. 8B is a schematic diagram of the positional relationship between the physiological signal sensing apparatus and the fabric of another embodiment of the present application.

FIG. 9A is a schematic diagram of the positional relationship among the physiological signal sensing apparatus and the behavior sensing apparatus and the fabric of an embodiment of the present application.

FIG. 9B is a schematic diagram of the positional relationship among the physiological signal sensing apparatus and the behavior sensing apparatus and the fabric of another embodiment of the present application.

FIG. 10A and FIG. 10B are respectively schematic diagrams of sensing electrodes in the capacitive coupling devices of different embodiments of the present application.

### DETAILED DESCRIPTION OF DISCLOSED EMBODIMENTS

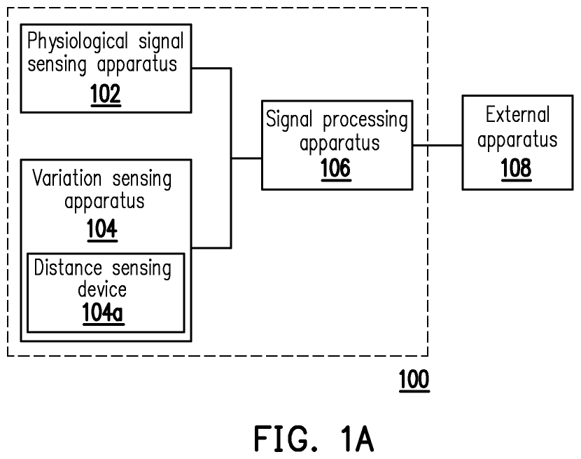

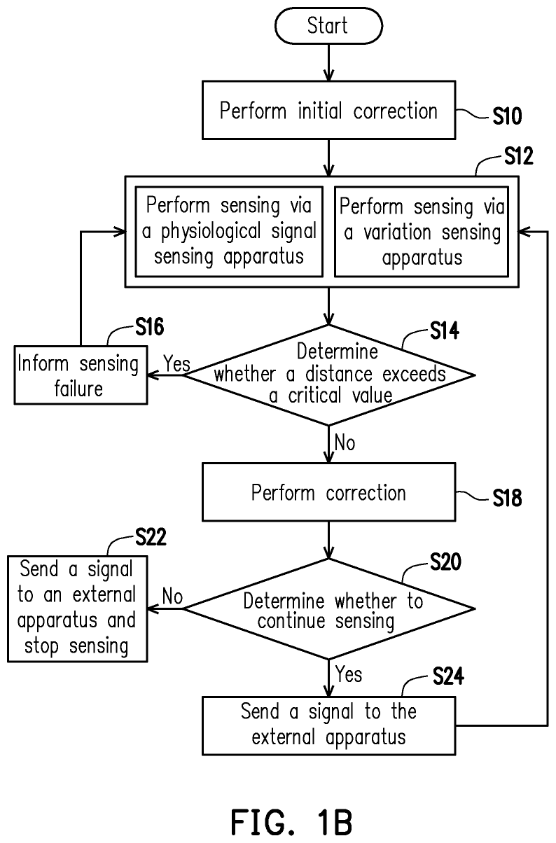

FIG. 1A is a block diagram of the physiological signal sensing system of the first embodiment of the present application. Referring to FIG. 1A, in the present embodiment, a physiological signal sensing system 100 includes a physiological signal sensing apparatus 102, a variation sensing apparatus 104, and a signal processing apparatus 106. The physiological signal sensing system 100 may sense the physiological signals of an organism in real time, and feedback the corrected physiological signals. Details are provided below.

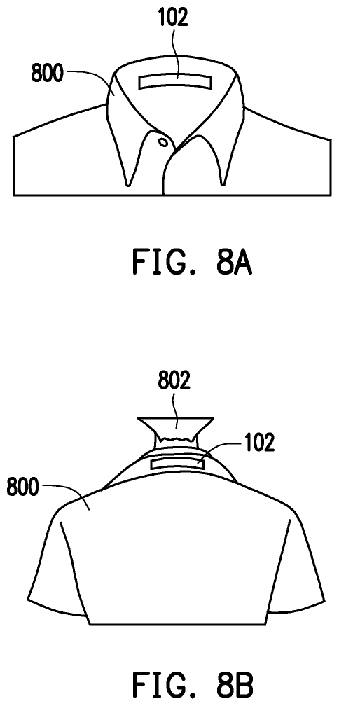

The physiological signal sensing apparatus 102 is disposed on a fabric. The physiological signal sensing apparatus 102 is configured to sense and provide the physiological signals of the organism. In the present embodiment, the organism is, for example, a human body, and the fabric is implemented in the form of, for example, clothing (such as coats, tops, pants, skirts, underwear), accessories (such as gloves, bracelets, anklets, hats, socks, belts, bandanas, cufflinks), patches, straps, waist protectors, knee protectors, ankle protectors and insoles that may be worn or put on by a user, mattresses, chair cushions, and the present application is not limited in this regard. In an embodiment of the present application, the physiological signal sensing apparatus 102 includes a plurality of capacitive coupling devices, which is described later. The physiological signals are, for example, electromyography (EMG) signals, electrocardiography (ECG) signals, or electroencephalography (EEG) signals. In addition, in the present embodiment, the physiological signal sensing apparatus 102 is disposed on the inside of the fabric, that is, the physiological signal sensing apparatus 102 is located between the fabric and the organism. Referring to FIG. 8A, FIG. 8A is a schematic diagram of the positional relationship between the physiological signal sensing apparatus and the fabric of an embodiment of the present application, wherein the physiological signal sensing apparatus 102 is disposed on the inside of a fabric 800 so as to be adjacent to the organism. In this way, the physiological signal sensing apparatus 102 may directly and unimpededly sense the skin of the organism and provide physiological signals.

The variation sensing apparatus 104 is disposed on the fabric. The variation sensing apparatus 104 includes a distance sensing device 104a to sense the distance between the physiological signal sensing apparatus 102 and the organism (for example, the distance between the physiological signal sensing apparatus 102 and human skin), and provide a capacitance variation signal according to the distance. The distance sensing device 104a is, for example, a capacitor, a time-of-flight (TOF) sensor, an inductor, or an infrared sensor. In detail, during the movement of the organism, the physiological signal sensing apparatus 102 located on the fabric has different distances from the organism with different movements. The variation sensing apparatus 104 may sense the change in the distance between the physiological signal sensing apparatus 102 and the organism in real time to calculate the capacitance variation amount caused by the difference in distance, and provide a capacitance variation signal.

In an embodiment, the variation sensing apparatus 104 provides the capacitance variation signal according to the distance between the physiological signal sensing apparatus 102 and the organism as follows. First, a database of data on the distance variation amount and capacitance variation amount of various distances between the physiological signal sensing apparatus 102 and the organism may be established in advance. The capacitance variation amount may be calculated by substituting the distance variation amount of various distances into the capacitance formula (C=(ε×A)/d, wherein C is the capacitance value, ε is the relative dielectric constant, A is the area of the capacitor plate, and d is the distance). Next, the corresponding capacitance variation amount is obtained from the database according to the distance between the physiological signal sensing apparatus 102 and the organism in real time.

In an embodiment, the physiological signal sensing apparatus 102 and the variation sensing apparatus 104 may be disposed on the fabric. In another embodiment, the physiological signal sensing apparatus 102 and the variation sensing apparatus 104 may be simultaneously disposed on a flexible substrate, and the flexible substrate is disposed on the fabric. In yet another embodiment, one of the physiological signal sensing apparatus 102 and the variation sensing apparatus 104 may be disposed on the flexible substrate, and the other of the physiological signal sensing apparatus 102 and the variation sensing apparatus 104 is disposed on the fabric.

The signal processing apparatus 106 is coupled to the physiological signal sensing apparatus 102 and the variation sensing apparatus 104 to receive the physiological signals provided by the physiological signal sensing apparatus 102 and the capacitance variation signal provided by the variation sensing apparatus 104, and corrects the physiological signals according to the received capacitance variation signal to obtain corrected physiological signals. Also, the corrected physiological signals are the real time and accurate physiological index when the organism is performing a movement. The signal processing apparatus is, for example, a micro-control unit (MCU). The correction method is, for example, calculating the received capacitance variation signal and physiological signals using an analysis algorithm. The signal processing apparatus 106 may be disposed on a fabric or other suitable positions, and the present application is not limited in this regard. In addition, the signal processing apparatus 106 may be coupled to an external apparatus 108 outside the physiological signal sensing system 100 to output the corrected physiological signals to the external apparatus 108. The external apparatus 108 may be a display apparatus (such as a mobile phone, a watch, a tablet computer, etc.) or a warning apparatus (such as a vibrator, an alarm, etc.), and the disclosure is not limited in this regard. In this way, the user may accurately adjust the organism itself or the movement of the organism in real time according to the signals provided by the external apparatus 108.

The operation of the physiological signal sensing system 100 of the present embodiment is described below.

FIG. 1B is a flowchart of the physiological signal sensing method of the first embodiment of the present application. Referring to both FIG. 1A and FIG. 1B, first, in step S10, initial calibration may be performed on the physiological signal sensing apparatus 102 and/or the variation sensing apparatus 104. That is, the physiological signal sensing apparatus 102 and the variation sensing apparatus 104 are reset.

Then, in step S12, the physiological signal sensing apparatus 102 senses an organism and provides the physiological signals of the organism, and at the same time, the variation sensing apparatus 104 senses the distance between the physiological signal sensing apparatus 102 and the organism and provides a capacitance variation signal according to the distance. In this step, the distance between the physiological signal sensing apparatus 102 and the organism is changed with different movements due to the movements performed by the organism. Therefore, the variation sensing apparatus 104 may sense the change in distance in real time and provide a capacitance variation signal according to the change in capacitance caused by the change in distance.

Next, in step S14, the signal processing apparatus 106 receives the physiological signals provided by the physiological signal sensing apparatus 102 and the capacitance variation signal provided by the variation sensing apparatus 104, and determines whether the distance between the physiological signal sensing apparatus 102 and the organism exceeds a critical value. The critical value depends on the sensing limit of the physiological signal sensing apparatus 102 used, and the present application is not limited in this regard.

When the signal processing apparatus 106 determines that the distance exceeds the critical value, the physiological signals sensed by the physiological signal sensing apparatus 102 are inaccurate or unable to be sensed. Therefore, the signal processing apparatus 106 sends out a sensing failure signal (step S16). At this time, step S12 is repeated, and as the organism continues to move, the physiological signal sensing apparatus 102 and the variation sensing apparatus 104 perform sensing again.

When the signal processing apparatus 106 determines that the distance does not exceed the critical value, the physiological signal sensing apparatus 102 may reliably sense the physiological signals of the organism. Therefore, in step S18, the signal processing apparatus 106 corrects the physiological signals provided by the physiological signal sensing apparatus 102 according to the capacitance variation signal provided by the variation sensing device 104 to obtain corrected physiological signals.

In addition, after the corrected physiological signals are obtained, in step S20, the signal processing apparatus 106 may determine whether to continue the physiological signal sensing according to the corrected physiological signals. When the signal processing apparatus 106 determines not to continue the physiological signal sensing based on the user's preset or other factors, the signal processing apparatus 106 may stop the operation of the physiological signal sensing apparatus 102 and the variation sensing apparatus 104 and send a signal to the external apparatus 108 (step S22), so that the user may accurately adjust the organism itself or the movement of the organism in real time via the notification of the external apparatus 108. When the signal processing apparatus 106 determines to continue the physiological signal sensing, the signal processing apparatus 106 may also send a signal to the external apparatus 108 (step S24), so that the user may accurately adjust the organism itself or the movement of the organism in real time via the notification of the external apparatus 108. Moreover, the physiological signal sensing apparatus 102 senses the organism again and provides the physiological signals of the organism, and at the same time, the variation sensing apparatus 104 senses the distance between the physiological signal sensing apparatus 102 and the organism, i.e., step S12 is repeated.

Via the physiological signal sensing method of the present embodiment, the user (such as the organism itself) may accurately know the physiological signals of the organism during movement in real time, and may adjust the organism itself or the movement of the organism in real time.

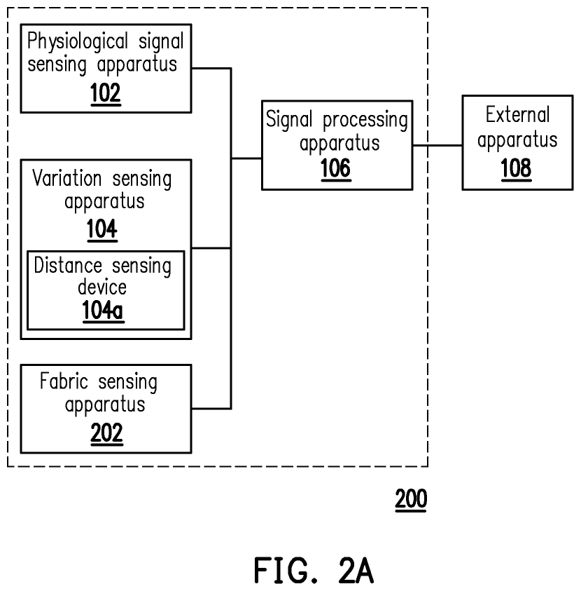

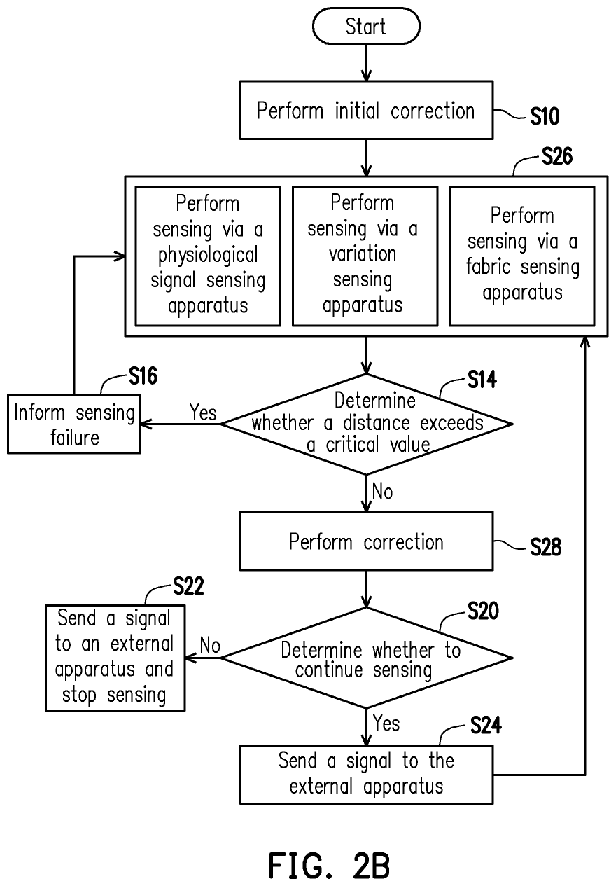

FIG. 2A is a block diagram of the physiological signal sensing system of the second embodiment of the present application. In the present embodiment, the same elements in FIG. 1A are labeled with the same reference numerals and are not repeated herein. Referring to FIG. 2A, in the present embodiment, a physiological signal sensing system 200 includes a physiological signal sensing apparatus 102, a variation sensing apparatus 104, a signal processing apparatus 106, and a fabric sensing apparatus 202. The physiological signal sensing system 200 may sense the physiological signals of the organism in real time, and further feedback the corrected physiological signals according to the characteristics of the fabric. Details are provided below.

The physiological signal sensing apparatus 102 is disposed on a fabric. In addition, in the present embodiment, the physiological signal sensing apparatus 102 is disposed on the outside of the fabric, that is, the fabric is located between the physiological signal sensing apparatus 102 and the organism. Referring to FIG. 8B, FIG. 8B is a schematic diagram of the positional relationship between the physiological signal sensing apparatus and the fabric of another embodiment of the present application, wherein the physiological signal sensing apparatus 102 is disposed on the outside of the fabric 800 so that the fabric 800 is located between the physiological signal sensing apparatus 102 and an organism 802. In this way, the physiological signal sensing apparatus 102 needs to sense the physiological signals of the organism with the fabric in between, and the sensed physiological signals are affected by the fabric.

The fabric sensing apparatus 202 is coupled to the signal processing apparatus 106. In the present embodiment, the fabric sensing apparatus 202 is disposed on a fabric, but the disclosure is not limited thereto. The fabric sensing apparatus 202 may be disposed at any suitable position, and may also be disposed on a flexible substrate simultaneously with the physiological signal sensing apparatus 102. The fabric sensing apparatus 202 may sense the dielectric constant of the fabric. The fabric sensing apparatus 202 is, for example, a capacitive device. After the fabric sensing apparatus 202 senses the dielectric constant of the fabric, the fabric sensing apparatus 202 may provide dielectric constant signals related to the dielectric constant to the signal processing apparatus 106. As a result, the signal processing apparatus 106 may receive the physiological signals provided by the physiological signal sensing apparatus 102, the capacitance variation signal provided by the variation sensing apparatus 104, and the dielectric constant signals provided by the fabric sensing apparatus 202, and correct the physiological signals according to the received capacitance variation signal and dielectric constant signals to obtain corrected physiological signals. Also, the corrected physiological signals are the real time and accurate physiological index when the organism is performing a movement.

The operation of the physiological signal sensing system 200 of the present embodiment is described below.

FIG. 2B is a flowchart of the physiological signal sensing method of the second embodiment of the present application. In the present embodiment, the same steps as those in the first embodiment are not specifically described herein. Referring to FIG. 2A and FIG. 2B at the same time, first, in step S10, initial calibration may be performed on one or more of the physiological signal sensing apparatus 102, the variation sensing apparatus 104, and the fabric sensing apparatus 202.

Then, in step S26, the physiological signal sensing apparatus 102 senses an organism and provides the physiological signals of the organism, and at the same time, the variation sensing apparatus 104 senses the distance between the physiological signal sensing apparatus 102 and the organism and provides a capacitance variation signal according to the distance, and the fabric sensing apparatus 202 senses the dielectric constant of the fabric and provides dielectric constant signals. In this step, the distance between the physiological signal sensing apparatus 102 and the organism is changed with different movements due to the movements performed by the organism.

Therefore, the variation sensing apparatus 104 may sense the change in distance in real time and provide a capacitance variation signal according to the change in capacitance caused by the change in distance. In addition, for various fabrics on the organism, dielectric constant signals affecting the physiological signals sensed may be provided according to the type of the fabric.

Next, in step S14, the signal processing apparatus 106 receives the physiological signals provided by the physiological signal sensing apparatus 102, the capacitance variation signal provided by the variation sensing apparatus 104, and the dielectric constant signals provided by the fabric sensing apparatus 202, and determines whether the distance between the physiological signal sensing apparatus 102 and the organism exceeds a critical value.

When the signal processing apparatus 106 determines that the distance exceeds the critical value, the signal processing apparatus 106 sends out a sensing failure signal (step S16). At this time, step S26 is repeated, and the physiological signal sensing apparatus 102 and the variation sensing apparatus 104 perform sensing again.

When the signal processing apparatus 106 determines that the distance does not exceed the critical value, in step S28, the signal processing apparatus 106 corrects the physiological signals provided by the physiological signal sensing apparatus 102 according to the capacitance variation signal provided by the variation sensing apparatus 104 and the dielectric constant signals provided by the fabric sensing apparatus 202 to obtain corrected physiological signals.

Then, as in the first embodiment, step S20 and step S22 or step S24 are performed. As a result, the user (such as the organism itself) may accurately know the physiological signals of the organism during movement in real time, and may adjust the organism itself or the movement of the organism in real time.

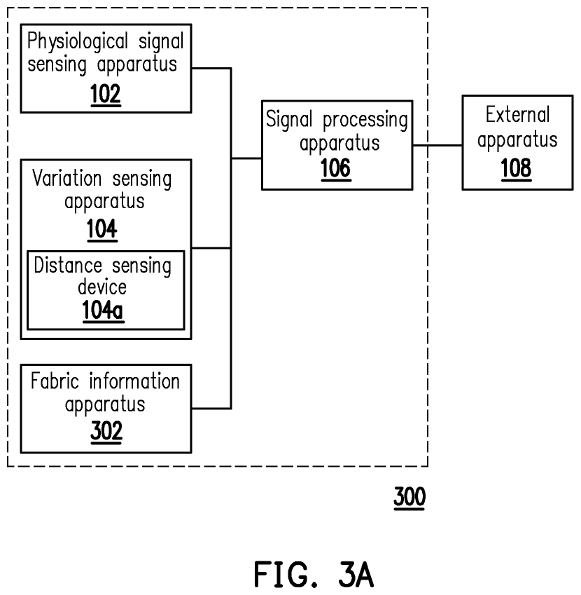

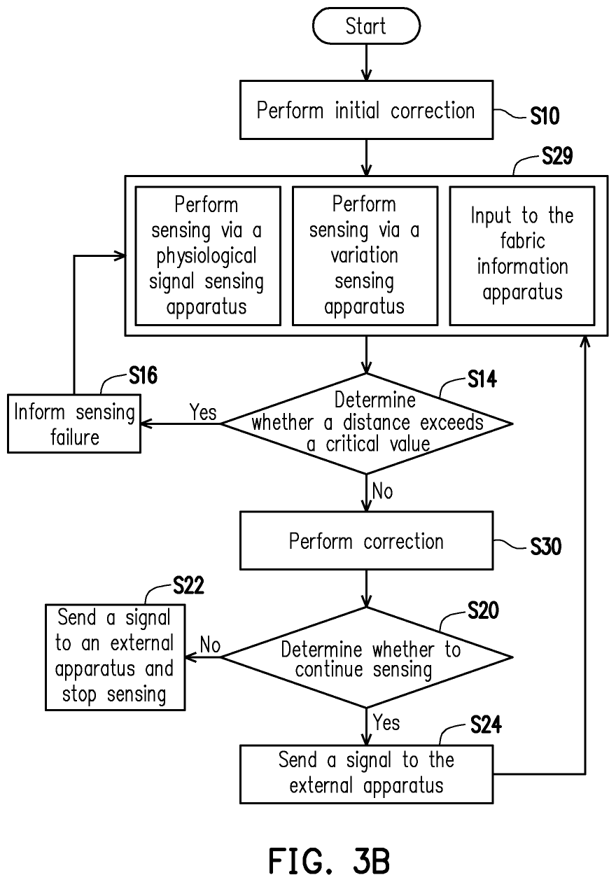

FIG. 3A is a block diagram of the physiological signal sensing system of the third embodiment of the present application. In the present embodiment, the same elements in FIG. 2A are labeled with the same reference numerals and are not repeated herein. Referring to FIG. 3A, in the present embodiment, a physiological signal sensing system 300 includes the physiological signal sensing apparatus 102, the variation sensing apparatus 104, the signal processing apparatus 106, and a fabric information apparatus 302. The physiological signal sensing system 300 may sense the physiological signals of the organism in real time, and further feedback the corrected physiological signals according to the characteristics of the fabric.

In the present embodiment, the difference between the physiological signal sensing system 300 and the physiological signal sensing system 200 is that the fabric sensing apparatus 202 in the physiological signal sensing system 200 is replaced with the fabric information apparatus 302. The fabric information apparatus 302 has a database storing dielectric constant information of various fabric materials. When the user inputs fabric material information to the fabric information apparatus 302, the fabric information apparatus 302 may obtain the dielectric constant corresponding to the fabric material from the database and provide the dielectric constant signals related to the dielectric constant to the signal processing apparatus 106. As a result, the signal processing apparatus 106 may correct the physiological signals according to the received capacitance variation signal and the dielectric constant signals to obtain corrected physiological signals, and the corrected physiological signals are the real time and accurate physiological index when the organism is performing a movement.

FIG. 3B is a flowchart of the physiological signal sensing method of the third embodiment of the present application. In the present embodiment, the same steps as those in the second embodiment are not specifically described herein. Referring to FIG. 3A and FIG. 3B at the same time, first, in step S10, initial calibration may be performed on the physiological signal sensing apparatus 102 and/or the variation sensing apparatus 104.

Then, in step S29, the physiological signal sensing apparatus 102 senses an organism and provides the physiological signals of the organism, and at the same time, the variation sensing apparatus 104 senses the distance between the physiological signal sensing apparatus 102 and the organism and provides a capacitance variation signal according to the distance. In addition, the user inputs the fabric material information to the fabric information apparatus 302, and the fabric information apparatus 302 obtains the dielectric constant corresponding to the fabric material from the database and provides dielectric constant signals. In this step, the distance between the physiological signal sensing apparatus 102 and the organism is changed with different movements due to the movements performed by the organism. Therefore, the variation sensing apparatus 104 may sense the change in distance in real time and provide a capacitance variation signal according to the change in capacitance caused by the change in distance. In addition, for various fabrics on the organism, dielectric constant signals affecting the physiological signals sensed may be provided according to the information of the fabric material input by the user.

Next, in step S14, the signal processing apparatus 106 receives the physiological signals provided by the physiological signal sensing apparatus 102, the capacitance variation signal provided by the variation sensing apparatus 104, and the dielectric constant signals provided by the fabric information apparatus 302, and determines whether the distance between the physiological signal sensing apparatus 102 and the organism exceeds a critical value.

When the signal processing apparatus 106 determines that the distance exceeds the critical value, the signal processing apparatus 106 sends out a sensing failure signal (step S16). At this time, step S28 is repeated, and the physiological signal sensing apparatus 102 and the variation sensing apparatus 104 perform sensing again.

When the signal processing apparatus 106 determines that the distance does not exceed the critical value, in step S30, the signal processing apparatus 106 corrects the physiological signals provided by the physiological signal sensing apparatus 102 according to the capacitance variation signal provided by the variation sensing apparatus 104 and the dielectric constant signals provided by the fabric information apparatus 302 to obtain corrected physiological signals.

Then, as in the second embodiment, step S20 and step S22 or step S24 are performed. As a result, the user (such as the organism itself) may accurately know the physiological signals of the organism during movement in real time, and may adjust the organism itself or the movement of the organism in real time.

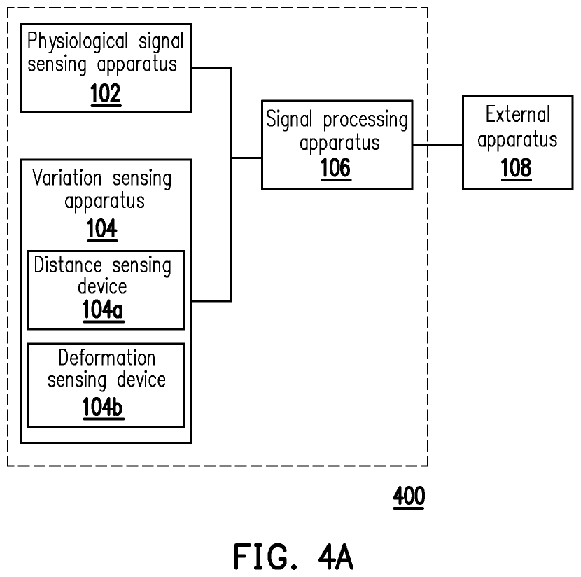

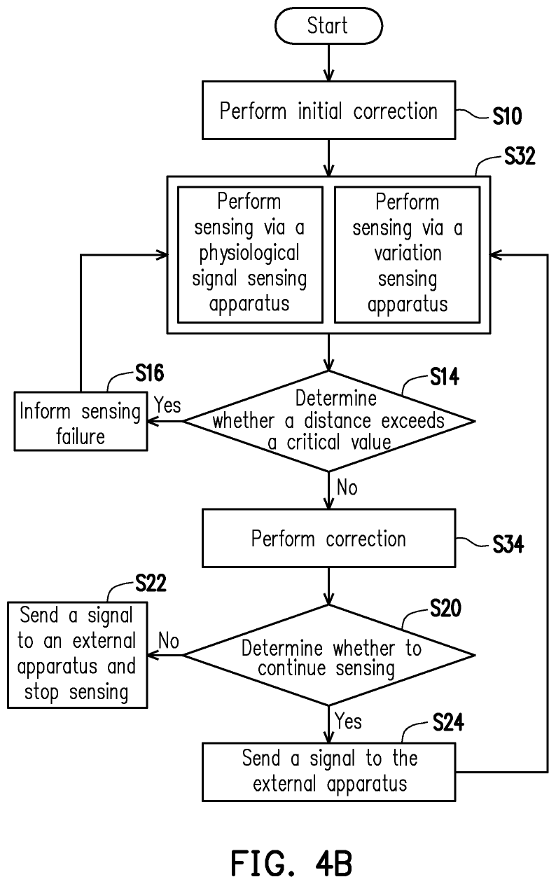

FIG. 4A is a block diagram of the physiological signal sensing system of the fourth embodiment of the present application. In the present embodiment, the same elements in FIG. 1A are labeled with the same reference numerals and are not repeated herein. Referring to FIG. 4A, in the present embodiment, a physiological signal sensing system 400 includes the physiological signal sensing apparatus 102, the variation sensing apparatus 104, and the signal processing apparatus 106, and the variation sensing apparatus 104 includes both the distance sensing device 104a and a deformation sensing device 104b. The physiological signal sensing system 400 may sense the physiological signals of the organism in real time, and further feedback the corrected physiological signals according to the characteristics of the fabric. Details are provided below.

In the present embodiment, the deformation sensing device 104b is disposed on the fabric, and is not limited to being located on the outside or inside of the fabric. The deformation sensing device 104b senses the bending curvature of the physiological signal sensing apparatus 102, and provides another capacitance variation signal according to the bending curvature. Since the physiological signal sensing apparatus 102 is disposed on the fabric, during the movement of the organism, the physiological signal sensing apparatus 102 located on the fabric is bent with the deformation of the fabric, so that the distance between the physiological signal sensing apparatus 102 and the organism is not uniform. The deformation sensing device 104b may sense the bending curvature of the physiological signal sensing apparatus 102 in real time to calculate the capacitance variation amount caused by the non-uniform distance, and provide a capacitance variation signal. In an embodiment, the distance sensing device 104a provides a first capacitance variation signal, and the deformation sensing device 104b provides a second capacitance variation signal. The first capacitance variation signal is different from the second capacitance variation signal. The deformation sensing device 104b is, for example, a capacitor or a resistor. In addition, in order to avoid mutual interference during sensing, preferably, the distance sensing device 104a and the deformation sensing device 104b may not be capacitors at the same time. As a result, the signal processing apparatus 106 may receive the physiological signals provided by the physiological signal sensing apparatus 102 and the two capacitance variation signals provided by the variation sensing apparatus 104 and correct the physiological signals according to the received two capacitance variation signals to obtain corrected physiological signals. Also, the corrected physiological signals are the real time and accurate physiological index when the organism is performing a movement.

The operation of the physiological signal sensing system 400 of the present embodiment is described below.

FIG. 4B is a flowchart of the physiological signal sensing method of the fourth embodiment of the present application. In the present embodiment, the same steps as those in the first embodiment are not specifically described herein. Referring to FIG. 4A and FIG. 4B at the same time, first, in step S10, initial calibration may be performed on the physiological signal sensing apparatus 102 and/or the variation sensing apparatus 104.

Then, in step S32, the physiological signal sensing apparatus 102 senses an organism and provides the physiological signals of the organism, and at the same time, the distance sensing device 104a in the variation sensing apparatus 104 senses the distance between the physiological signal sensing apparatus 102 and the organism and provides a first capacitance variation signal, and the deformation sensing device 104b in the variation sensing apparatus 104 senses the bending curvature of the physiological signal sensing apparatus 102 and provides a second capacitance variation signal. In this step, the distance between the physiological signal sensing apparatus 102 and the organism is changed with different movements due to the movements performed by the organism, and the physiological signal sensing apparatus 102 is bent with the deformation of the fabric. Therefore, the variation sensing apparatus 104 including the distance sensing device 104a and the deformation sensing device 104b senses the change in distance and the bending curvature change in real time, and provides two capacitance variation signals according to the change in capacitance caused by these changes.

Next, in step S14, the signal processing apparatus 106 receives the physiological signals provided by the physiological signal sensing apparatus 102 and the two capacitance variation signals provided by the variation sensing apparatus 104, and determines whether the distance between the physiological signal sensing apparatus 102 and the organism exceeds a critical value.

When the signal processing apparatus 106 determines that the distance exceeds the critical value, the signal processing apparatus 106 sends out a sensing failure signal (step S16). At this time, step S32 is repeated, and the physiological signal sensing apparatus 102 and the variation sensing apparatus 104 perform sensing again.

When the signal processing apparatus 106 determines that the distance does not exceed the critical value, in step S34, the signal processing apparatus 106 corrects the physiological signals provided by the physiological signal sensing apparatus 102 according to the first capacitance variation signal and the second capacitance variation signal provided by the variation sensing apparatus 104 to obtain corrected physiological signals.

Then, as in the first embodiment, step S20 and step S22 or step S24 are performed. As a result, the user (such as the organism itself) may accurately know the physiological signals of the organism during movement in real time, and may adjust the organism itself or the movement of the organism in real time.

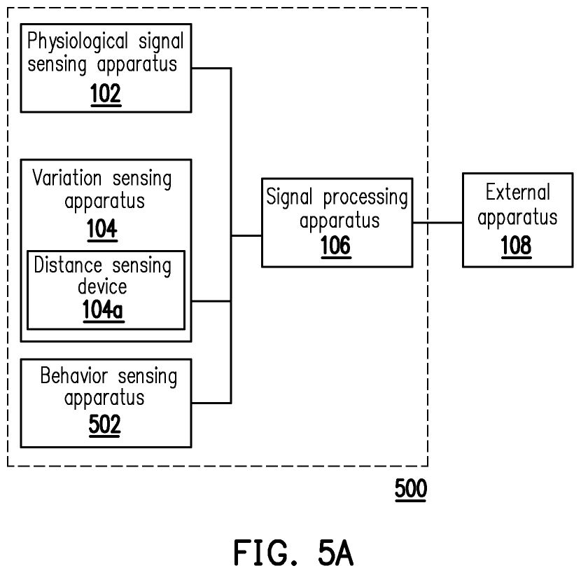

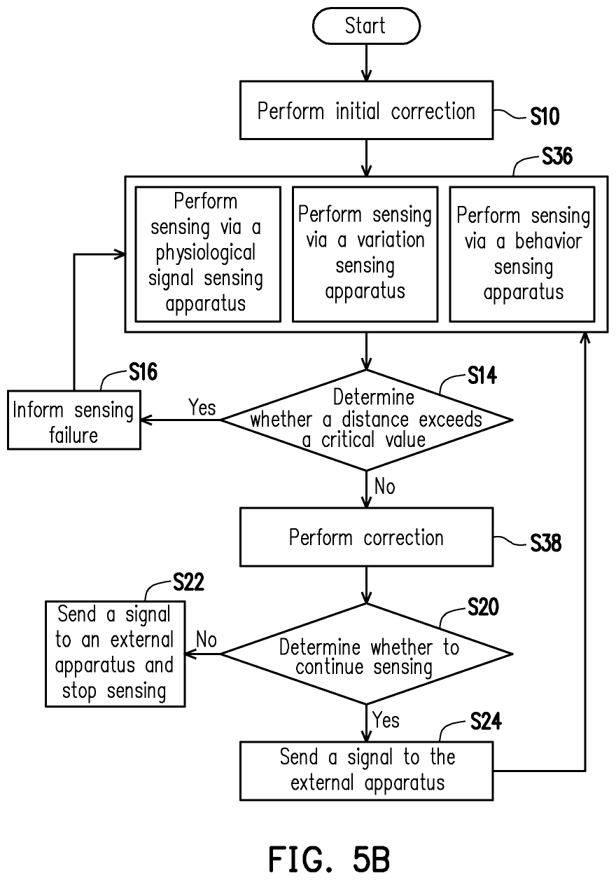

FIG. 5A is a block diagram of the physiological signal sensing system of the fifth embodiment of the present application. In the present embodiment, the same elements in FIG. 1A are labeled with the same reference numerals and are not repeated herein. Referring to FIG. 5A, in the present embodiment, a physiological signal sensing system 500 includes the physiological signal sensing apparatus 102, the variation sensing apparatus 104, the signal processing apparatus 106, and a behavior sensing apparatus 502. The physiological signal sensing system 500 may sense the physiological signals and behavior information of the organism in real time. Details are provided below.

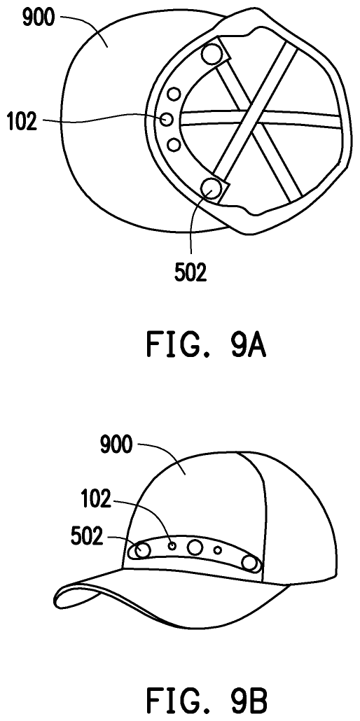

The behavior sensing apparatus 502 is coupled to the signal processing apparatus 106. In the present embodiment, the behavior sensing apparatus 502 is disposed on a fabric, but the disclosure is not limited thereto. The behavior sensing apparatus 502 may be disposed at any suitable position. The behavior sensing apparatus 502 is, for example, an accelerometer, a G-sensor, or a pressure sensor. The behavior sensing apparatus 502 senses the behavior pattern of the organism (for example, the posture of the organism, the duration of movement, etc.), and provides behavior signals related to the sensed behavior pattern to the signal processing apparatus 106. As a result, the signal processing apparatus 106 may receive the physiological signals provided by the physiological signal sensing apparatus 102, the capacitance variation signal provided by the variation sensing apparatus 104, and the behavior signals provided by the behavior sensing apparatus 502, and correct the physiological signals according to the received capacitance variation signals and behavior signals to obtain corrected physiological signals. Also, the corrected physiological signals are the real time and accurate physiological index when the organism is performing a movement. FIG. 9A is a schematic diagram of the positional relationship between the physiological signal sensing apparatus and the behavior sensing apparatus and the fabric of an embodiment of the present application. As shown in FIG. 9A, the physiological signal sensing apparatus 102 and the behavior sensing apparatus 502 are disposed on the inside of a fabric 900 (such as a hat) so as to be adjacent to the organism. FIG. 9B is a schematic diagram of the positional relationship between the physiological signal sensing apparatus and the behavior sensing apparatus and the fabric of another embodiment of the present application. As shown in FIG. 9B, the physiological signal sensing apparatus 102 and the behavior sensing apparatus 502 are disposed on the outside of the fabric 900 such that the fabric 900 is located between the organism and the physiological signal sensing apparatus 102 and the behavior sensing apparatus 502.

The operation of the physiological signal sensing system 500 of the present embodiment is described below.

FIG. 5B is a flowchart of the physiological signal sensing method of the fifth embodiment of the present application. In the present embodiment, the same steps as those in the first embodiment are not specifically described herein. Referring to FIG. 5A and FIG. 5B at the same time, first, in step S10, initial calibration may be performed on one or more of the physiological signal sensing apparatus 102, the variation sensing apparatus 104, and the behavior sensing apparatus 502.

Then, in step S36, the physiological signal sensing apparatus 102 senses an organism and provides the physiological signals of the organism, and at the same time, the variation sensing apparatus 104 senses the distance between the physiological signal sensing apparatus 102 and the organism and provides a capacitance variation signal according to the distance, and the behavior sensing apparatus 502 senses the behavior pattern of the organism and provides behavior signals. In this step, the distance between the physiological signal sensing apparatus 102 and the organism is changed with different movements due to the movements performed by the organism. Therefore, the variation sensing apparatus 104 may sense the change in distance in real time and provide a capacitance variation signal according to the change in capacitance caused by the change in distance. In addition, for various movements on the organism, behavior signals affecting the sensed physiological signals may be provided according to behavior patterns.

Next, in step S14, the signal processing apparatus 106 receives the physiological signals provided by the physiological signal sensing apparatus 102, the capacitance variation signal provided by the variation sensing apparatus 104, and the behavior signals provided by the behavior sensing apparatus 502, and determines whether the distance between the physiological signal sensing apparatus 102 and the organism exceeds a critical value.

When the signal processing apparatus 106 determines that the distance exceeds the critical value, the signal processing apparatus 106 sends out a sensing failure signal (step S16). At this time, step S36 is repeated, and the physiological signal sensing apparatus 102, the variation sensing apparatus 104, and the behavior sensing apparatus 502 perform sensing again.

When the signal processing apparatus 106 determines that the distance does not exceed the critical value, in step S38, the signal processing apparatus 106 corrects the physiological signals provided by the physiological signal sensing apparatus 102 according to the capacitance variation signal provided by the variation sensing apparatus 104 and the behavior signals provided by the behavior sensing apparatus 502 to obtain corrected physiological signals.

In the present embodiment, before the behavior signals are provided to the signal processing apparatus 106, the noise in the behavior signals not affecting the sensed physiological signals may be removed by a filter.

Then, as in the first embodiment, step S20 and step S22 or step S24 are performed. As a result, the user (such as the organism itself) may accurately know the physiological signals of the organism during movement in real time, and may adjust the organism itself or the movement and behavior of the organism in real time. In addition, in the present embodiment, the behavior signals provided by the behavior sensing apparatus 502 may provide behavior pattern analysis information of the organism.

In each embodiment of the present application, in addition to including the physiological signal sensing apparatus 102, the variation sensing apparatus 104 including the distance sensing device 104a, and the signal processing apparatus 106, the physiological signal sensing system may be optionally provided with at least one of the fabric sensing apparatus 202, the fabric information apparatus 302, the deformation sensing device 104b, and the behavior sensing apparatus 502 based on actual needs. That is, the present application is not limited to the first embodiment to the fifth embodiment above.

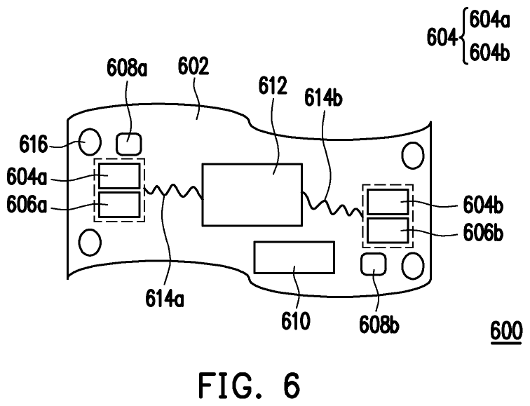

FIG. 6 is a schematic diagram of the device configuration of the physiological signal sensing system of an embodiment of the present application. In the present embodiment, a physiological signal sensing system 600 is disposed on a flexible substrate 602 and includes a physiological signal sensing apparatus 604 with capacitive coupling devices 604a and 604b, variation sensing apparatuses 606a and 606b, behavior sensing apparatuses 608a and 608b, a battery 610, a signal processing apparatus 612, and circuits 614a and 614b (such as stretchable circuits). In another embodiment, the physiological signal sensing system may further include a Bluetooth apparatus (not shown) according to actual needs. The capacitive coupling device 604a and the variation sensing apparatus 606a are coupled to the signal processing apparatus 612 via a circuit 614a. The capacitive coupling device 604b and the variation sensing apparatus 606b are coupled to the signal processing apparatus 612 via a circuit 614b. The behavior sensing apparatuses 608a and 608b may be located at suitable positions on the flexible substrate 602 and coupled with the signal processing apparatus 612. The battery 610 may provide energy to the physiological signal sensing system 600. In addition, the physiological signal sensing system 600 may further include a fixing apparatus 616 provided on the flexible substrate 602 to fix the flexible substrate 602 on the fabric. In the present embodiment, the physiological signal sensing system 600 includes two variation sensing apparatuses, but the present application is not limited thereto. In other embodiments, the physiological signal sensing system may include one variation sensing apparatus.

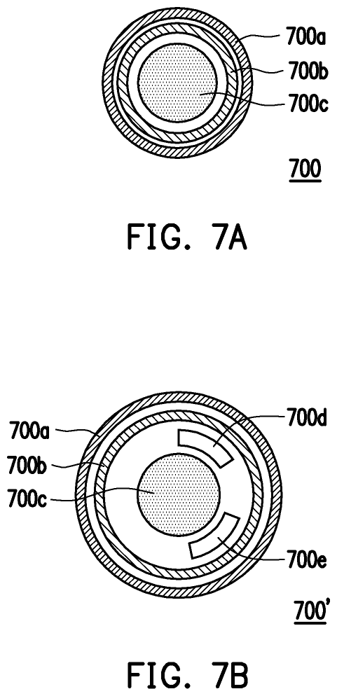

In addition, the capacitive coupling devices 604a and 604b may have an architecture as shown in FIG. 7A or FIG. 7B, but the present application is not limited thereto. In other embodiments, the capacitive coupling devices may adopt other architectures according to actual needs. FIG. 7A and FIG. 7B are respectively schematic diagrams of capacitive coupling devices of different embodiments of the present application. As shown in FIG. 7A, a capacitive coupling device 700 may include a grounding circuit 700a, a shielding circuit 700b, and a sensing electrode 700c. In the present embodiment, the shielding circuit 700b is located between the grounding circuit 700a and the sensing electrode 700c, but the present application is not limited thereto. In another embodiment, the grounding circuit 700a may be located between the shielding circuit 700b and the sensing electrode 700c. Moreover, as shown in FIG. 7B, a capacitive coupling device 700′ may include the grounding circuit 700a, the shielding circuit 700b, the sensing electrode 700c, a filter 700d, and an amplifier 700e. The sensing electrode 700c is coupled to the filter 700d and the amplifier 700e, that is, the capacitive coupling device 700′ may include the sensing electrode 700c, the filter 700d, and the amplifier 700e electrically connected to one another. In the present embodiment, the shielding circuit 700b is located between the grounding circuit 700a and the sensing electrode 700c, but the present application is not limited thereto. In another embodiment, the grounding circuit 700a may be located between the shielding circuit 700b and the sensing electrode 700c. The grounding circuit 700a and the shielding circuit 700b may preliminarily filter out signals to remove interference. The preliminarily filtered signals may be amplified by the amplifier 700e, and the amplified signals may be filtered by the filter 700d for a second time. In an embodiment of the disclosure, the filter 700d and the amplifier 700e are disposed in the capacitive coupling device 700′, so when the physiological signal sensing apparatus senses the physiological signals, the signals may be amplified and filtered.

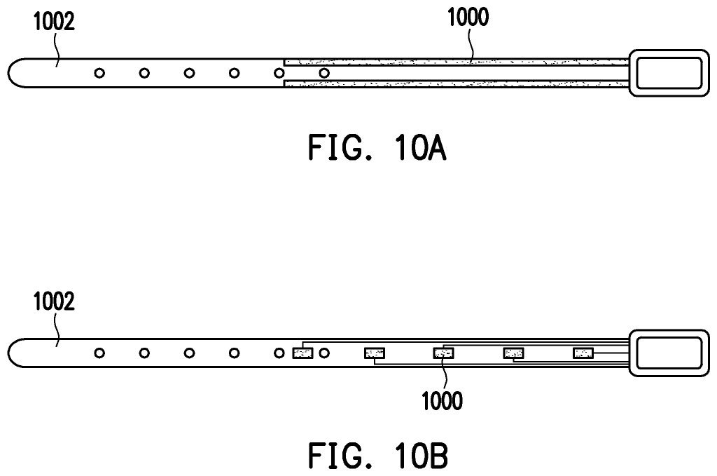

In an embodiment, when the shape of the fabric is a long strip (the fabric is a leather belt or a seat belt, for example), the sensing electrode may be a long strip electrode or a plurality of segment-shaped electrodes disposed in parallel. FIG. 10A and FIG. 10B are respectively schematic diagrams of sensing electrodes in the capacitive coupling devices of different embodiments of the present application. As shown in FIG. 10A, a long strip sensing electrode 1000 is disposed on a leather belt 1002. Alternatively, as shown in FIG. 10B, a plurality of segment-shaped sensing electrodes 1000 are connected to each other in parallel and disposed on the leather belt 1002.

The physiological signal sensing system of each embodiment of the present application may adopt a device configuration similar to that shown in FIG. 6 according to actual conditions, but the present application is not limited in this regard.

In addition, in each embodiment of the present application, the physiological signal sensing system may further include a physiological value detection apparatus or an apparatus detection device. For example, the physiological signal sensing system of the present application may also include a blood sugar detector, a calorie detector, a weight detector, and so on. In addition, the physiological signal sensing system of the present application may also include a gyro sensor to determine the positioning status of sensing apparatuses such as the physiological signal sensing apparatus 102 and the variation sensing apparatus 104, and provide positioning information for the user to adjust the position of each sensing apparatus in real time, so as to reduce or avoid the sensing error of the physiological signal sensing system of the present application.

It will be apparent to those skilled in the art that various modifications and variations may be made to the structure of the disclosed embodiments without departing from the scope or spirit of the disclosure. In view of the foregoing, it is intended that the disclosure cover modifications and variations of this disclosure provided they fall within the scope of the following claims and their equivalents.

What is claimed is:

1. A physiological signal sensing system, comprising:
a physiological signal sensing apparatus disposed on a fabric to sense and provide physiological signals of an organism, wherein the physiological signal sensing apparatus comprises capacitive coupling devices;
a variation sensing apparatus disposed on the fabric and comprising a distance sensing device to sense a distance between the physiological signal sensing apparatus and the organism and provide a first capacitance variation signal according to the distance; and
a signal processing apparatus coupled to the physiological signal sensing apparatus and the variation sensing apparatus to receive the physiological signals and the first capacitance variation signal and perform a correction on the physiological signals according to the first capacitance variation signal to obtain corrected physiological signals.

2. The physiological signal sensing system of claim 1, wherein the physiological signals comprise electromyography (EMG) signals, electrocardiography (ECG) signals, or electroencephalography (EEG) signals.

3. The physiological signal sensing system of claim 1, wherein the distance sensing device comprises a capacitor, a time-of-flight sensor, an inductor, or an infrared sensor.

4. The physiological signal sensing system of claim 1, wherein the fabric is located between the physiological signal sensing apparatus and the organism, the physiological signal sensing system further comprises a fabric sensing apparatus coupled to the signal processing apparatus, the fabric sensing apparatus senses a dielectric constant of the fabric and provides dielectric constant signals, and the signal processing apparatus performs the correction on the physiological signals according to the first capacitance variation signal and the dielectric constant signals.

5. The physiological signal sensing system of claim 1, wherein the fabric is located between the physiological signal sensing apparatus and the organism, the physiological signal sensing system further comprises a fabric information apparatus coupled to the signal processing apparatus, the fabric information apparatus provides dielectric constant signals related to a dielectric constant of the fabric, and the signal processing apparatus performs the correction on the physiological signals according to the first capacitance variation signal and the dielectric constant signals.

6. The physiological signal sensing system of claim 1, wherein the physiological signal sensing apparatus is located between the fabric and the organism.

7. The physiological signal sensing system of claim 1, wherein the variation sensing apparatus further comprises a deformation sensing device to sense a bending curvature of the physiological signal sensing apparatus and provide a second capacitance variation signal according to the bending curvature, and the signal processing apparatus performs a physiological signal correction according to the first capacitance variation signal and the second capacitance variation signal to obtain the corrected physiological signals.

8. The physiological signal sensing system of claim 7, wherein the deformation sensing device comprises a capacitor or a resistor.

9. The physiological signal sensing system of claim 1, further comprising a behavior sensing apparatus coupled to the signal processing apparatus, wherein the behavior sensing apparatus senses a behavior pattern of the organism and provides behavior signals, and the signal processing apparatus performs the correction according to the first capacitance variation signal and the behavior signals.

10. The physiological signal sensing system of claim 1, wherein the capacitive coupling devices comprise a sensing electrode, a filter, and an amplifier electrically connected to one another.

11. A physiological signal sensing method, comprising:
sensing via a physiological signal sensing apparatus disposed on a fabric and providing physiological signals of an organism, wherein the physiological signal sensing apparatus comprises capacitive coupling devices;
sensing a distance between the physiological signal sensing apparatus and the organism via a variation sensing apparatus comprising a distance sensing device disposed on the fabric, and providing a first capacitance variation signal according to the distance; and
receiving the physiological signals and the first capacitance variation signal via a signal processing apparatus coupled to the physiological signal sensing apparatus and the variation sensing apparatus, and performing a correction on the physiological signals according to the first capacitance variation signal to obtain corrected physiological signals.

12. The physiological signal sensing method of claim 11, wherein the signal processing apparatus determines whether the distance does not exceed a critical value before the correction is performed.

13. The physiological signal sensing method of claim 12, wherein when the distance exceeds the critical value, the signal processing apparatus does not perform the correction.

14. The physiological signal sensing method of claim 12, wherein when the distance does not exceed the critical value, the signal processing apparatus performs the correction.

15. The physiological signal sensing method of claim 11, wherein when the fabric is located between the physiological signal sensing apparatus and the organism, a dielectric constant of the fabric is sensed and dielectric constant signals are provided via a fabric sensing apparatus coupled to the signal processing apparatus, and the signal processing apparatus performs the correction according to the first capacitance variation signal and the dielectric constant signals.

16. The physiological signal sensing method of claim 11, wherein when the fabric is located between the physiological signal sensing apparatus and the organism, dielectric constant signals related to a dielectric constant of the fabric are provided via a fabric information apparatus coupled to the signal processing apparatus, and the signal processing apparatus performs the correction on the physiological signals according to the first capacitance variation signal and the dielectric constant signals.

17. The physiological signal sensing method of claim 11, wherein the variation sensing apparatus further comprises a deformation sensing device, the physiological signal sensing method further comprises sensing a bending curvature of the physiological signal sensing apparatus and providing a second capacitance variation signal according to the bending curvature via the variation sensing apparatus, and the signal processing apparatus performs the correction according to the first capacitance variation signal and the second capacitance variation signal.

18. The physiological signal sensing method of claim 11, further comprising sensing a behavior pattern of the organism and providing behavior signals via a behavior sensing apparatus coupled to the signal processing apparatus, wherein the signal processing apparatus performs the correction according to the first capacitance variation signal and the behavior signals.

19. The physiological signal sensing method of claim 11, wherein the capacitive coupling devices comprise a sensing electrode, a filter, and an amplifier electrically connected to one another.
